(19) United States
(12) Patent Application Publication
(10) Pub. No.: US 2011/0256588 A1
(43) Pub. Date: Oct. 20, 2011

(54) METHOD OF MANUFACTURING REBAUDIOSIDE A IN HIGH YIELD BY RECYCLING BY-PRODUCTS PRODUCED FROM MANUFACTURING PROCESS FOR REBAUDIOSIDE A

(75) Inventors: Young Mi Lee, Bucheon-si (KR); Seong Bo Kim, Seoul (KR); Young Ho Hong, Gwangmyeong-si (KR); Joo Hang Lee, Ansan-si (KR); Seung Won Park, Yongin-si (KR)

(73) Assignee: CJ CHEILJEDANG CORPORATION, Seoul (KR)

(21) Appl. No.: 13/089,245

(22) Filed: Apr. 18, 2011

(30) Foreign Application Priority Data
Apr. 16, 2010 (KR) 10-2010-0035163

(51) Int. Cl. C12P 19/56

(52) U.S. Cl. 435/78

(57) ABSTRACT

The present invention relates to a method of producing Rebaudioside A in a high yield by recycling by-products produced when Rebaudioside A is produced from leaves of Stevia Rebaudiana Bertoni containing a sweetening material.

### BACKGROUND OF THE INVENTION

1. Field of the Invention

The present disclosure relates to a method of manufacturing Rebaudioside A in a high yield and high purity by reusing by-products produced from a manufacturing process for Rebaudioside A. More particularly, the present disclosure provides Rebaudioside A in a higher yield and higher purity than that of the prior manufacturing method, by reusing by-products, which are produced from a crystallization process of Rebaudioside A, as a raw material.

More particularly, the present disclosure provides a manufacturing method of Rebaudioside A, which comprises the steps enhancing the purity of mother liquor of Rebaudioside A crystals by eliminating materials besides steviol glycosides such as minerals, ash, and other organic materials contained therein through re-crystallization; and converting stevioside, which is a major ingredient, into Rebaudioside A by a conversion method using a microorganism or enzyme with β-1,3-glucosyl transactivation so that it can be reused as a raw material

2. Discussion of the Background

Stevioside (ST), which is contained in a Stevia plant, is a diterpene glycoside having steviol as aglycon, and besides stevioside other sweetening ingredients are Rebaudiosides A, C, D, E, and Dulcoside A. Such sweetening components are different in their degrees of sweetness, and although a relationship between sweetness and a characteristic in chemical structure has not been clearly revealed, it has been known that sweetness and quality of sweetness are largely affected by a glucose bonding site of a glycoside, an arrangement of functional groups (in particular, —OH), and a inter-distance of the arrangement thereof. Stevioside and Rebaudioside A (β-1,3-monoglucosyl stevioside) are high intensity natural sweeteners having sweetness of about 200 and 250 times higher than that of sugar, respectively. Stevioside leaves a bit of bitter taste behind, whereas Rebaudioside A has almost no bitter taste, and thus is superior in its sweetening properties. Likewise, Rebaudioside A, which is superior in sweetness and sweetening properties, is drawing much attention as a high intensity natural sweetener that can replace a current high intensity synthesized sweetener. In particular, the US FDA has approved only stevioside that contains 95% or more of Rebaudioside A as a high intensity sweetener and that can be used as a food additive since 2008. Thereafter, large food companies in the U.S.A. are actively using Rebaudioside A, and Rebaudioside A has already been commercially available in the trade name of PureVia and TRUVIA. Among these products, a leading product in the stevia market, TRUVIA is a product produced by Cargill and Coca-Cola, and occupies 58% of the stevia market. Yet, the market share of TRUVIA in the artificial sugar market is only 6%. This is because the manufacturing cost for TRUVIA is high and TRUVIA gives of a very unique flavor. Accordingly, many food companies are making efforts to overcome such problems.

A conventional production process of Rebaudioside A can be largely divided into two steps. The first step is the step for obtaining a purified product of steviol glycoside with high content, wherein the purified steviol glycoside in the first step is conventionally used as a high intensity sweetener in the Southeast Asia market. The first step produces the product by the following processes: extracting a solution containing steviol glycoside from dry stevia leaves by using a hydrothermal fluid, ethanol, methanol, or polyalcohols; decolorizing a pigment and so on that are contained in the extracted solution; purifying the decolorized solution by desalinization, microfiltration and adsorbing resin to give steviol glycoside in a high purity; and spraying and drying, etc. the obtained steviol glycoside. (see FIG. 1).

However, since a steviol glycoside content ratio in a product produced from the first step remains the same as that in the dry stevia leaves raw material, substantially, a Rebaudioside A content ratio in a final product is as low as 20% or as high as 60%, and the content ratio in the final product is dependently varying according to a seed of stevia cultivated as a raw material and a cultivation condition. Conventionally, a high purity steviol glycoside product produced as described above is directly used as a high intensity sweetener as itself, or as an enzyme treated-product produced by purification through a glucose-transferring enzymatic reaction.

The second step is a selective isolation and purification step for enhancing the purity of Rebaudioside A. A high purity product produced through the second step is conventionally limitedly produced and sold for the purpose of sales in such as the U.S.A. and Europe, where only a high purity product is approved for use as a food additive. Such second step, a process using a selective fractional crystallization principle is performed using a high purity product of steviol glycoside that has been produced (or sold) through the first step as a raw material. According to a conventional example of the second step, 50 to 60% of Rebaudioside A as a raw material in a mixed solution including alcohols (EtOH) is collected through crystallization preparing 80 to 85% of Rebaudioside A as a primary crystallization product, and then the 80 to 85% of Rebaudioside A is dissolved in a solvent having a higher alcohol (EtOH) content than the above and then crystallized, thereby finally producing 95% or higher of Rebaudioside A as a high purity product (see FIG. 1).

A fractional crystallization process has an advantageous feature of purifying a single material in order to produce a high purity among other materials having similar properties. However, it has a physical limitation in that the obtain-ratio of target product obtainable from one-time crystal collection is low. In particular, in the case of high purity Rebaudioside A, due to the characteristic of the typical process using two-time crystallization as a major process, it is difficult to increase the common obtain-ratio of Rebaudioside A beyond 50%.

After all, during the process of producing a high purity Rebaudioside A product as described above, more by-products are obtained than the target product. The by-products are sold as a first raw material for enzyme processed products at low product value, and it can be found that the characteristic of the by-product is a major factor limiting profitability in the overall process steps to produce Rebaudioside A beginning with stevis dry leaves.

In order to overcome such problems, there have been efforts to cultivate a plant species having a high Rebaudioside A content so as to produce Rebaudioside A in a great amount, and seeds that were proven to be partially effective are disclosed in various prior arts including Korean Patent No. 10-2008-0058236 (Title of the Invention: New Species of Stevia Plant containing High Content Rebaudioside-A and Cultivation Method Thereof), and overseas patents including PCT/JP2006/303992, US00PP10562P (Title of the Invention: Stevia Plant Named “RSIT 94-1306”, US00PP10563P (Title of the Invention: Stevia Plant Named “RSIT 95-166-13”, and US00PP10564P (Title of the Invention: Stevia Plant Named “RSIT 97-751”.)

However, a stevia plant requires as long as 5 to 6 months from sowing to harvesting, and a wide area. In particular, its yield is dependent upon weather conditions every year, and production cost is determined according to a cultivation environment and labor costs, and product quality is not uniform. Accordingly, there is a limitation in producing a Rebaudioside A with high content product through plant breeding, in aspects of production costs, production amount, quality, etc.

Meanwhile, researchers, in particular, some Japanese researchers performed various studies on an enzyme transferring technique for increasing the Rebaudioside A content in steviol glycoside.

The objective of the studies performed by the researchers is to artificially increase the content of Rebaudioside A having good sweetening quality and a high degree of perceived sweetness in steviol glycoside, thereby increasing the added value of a product. More recently, researchers carried out a study for increasing the obtain-ratio of Rebaudioside A by processing a primary raw material that mainly contains stevioside by using an enzyme transferring technique to increase the obtain-ratio of Rebaudioside A, and ultimately, to reduce manufacturing cost. In particular, Dainppon Ink and Chemicals, Inc., has been granted a patent right for an invention related to such study results (see U.S. Pat. No. 4,590,160). The US patent discloses a process of producing Rebaudioside A, which comprises reacting stevioside with a β-1,3-glycosyl sugar compound in an aqueous solution or an aqueous suspension in the presence of a microorganism or enzyme having β-1,3-glycosyl transferring activity thereby to form Rebaudioside A.

However, the method also does not provide a satisfactory purity level. Accordingly, there is a need to develop a method of manufacturing high purity Rebaudioside A.

### SUMMARY OF THE INVENTION

The present invention relates to a method of producing Rebaudioside A in a high yield and high purity, and in particular, relates to a Rebaudioside A production method in which by-products produced from a conventional manufacturing process for Rebaudioside A, particularly, residual by-products as a mother liquor produced from a fractional crystallization process are used as a starting material and subjected to a series of processes to have a reusable level suitable for the second step production process (high purity Rebaudioside A production process), and then the resultant by-products are re-circulated in the production process for Rebaudioside A. In particular, residual by-products are subjected to a series of purification processes, and crystallized to have a reusable level by using an enzyme transferring method so as to economically increase the Rebaudioside A content.

A prior art has been developed and applied simply to increase a Rebaudioside A content in a stevia raw material or a stevioside product whereas the present invention has a technical feature developing a re-circulation process in order to increase the obtain-ratio of the manufacturing process and providing a product with high purity by processing mother liquor by-product from cyristallization to a reusable level. According to the present invention, it has been found that it is more effective in respects of yield and purity to increase a Rebaudioside A content by using by-products produced from a first fractional crystallization process for a stevioside product than to increase a Rebaudioside A content in the stevioside product.

More particularly, method of the present invention comprises steps purifying a mother liquor produced from crystallization to increase a purified solution containing a steviol glycoside content therein to 90% or higher; adding an insoluble β-1,3 glucan, such as curdlan, to the purified solution; breaking down a β-1,3 bond of the β-1,3 glucan by β-1,3-glucanase to give a glucose; and connecting the glucose with the stevioside contained in the purified solution by using a specific glucosyl transferase to produce a Rebaudioside A of which content is 50% or higher.

A high content Rebaudioside A according to the prevent invention is prepared from either the product produced by such steps alone or a mixture of the product and a conventional raw material.

The inventors of the present invention have reached the completion of the invention by producing substantially high purity Rebaudioside A by setting stevioside contained in residual by-products produced after the extracted Rebaudioside A was purified and crystallized as an acceptor, and then by applying a β-1,3-glucosyl transferase that transfers glucose and also is an enzyme suitable for producing Rebaudioside A and a β-1,3-glucanase for breaking down β-1,3 glucose contained in a β-1,3-glucosyl oligosaccharide compound.

### BRIEF DESCRIPTION OF THE DRAWINGS

FIG. 1 is a flowchart illustrating a process of manufacturing Rebaudioside A, according to an embodiment of the present invention.

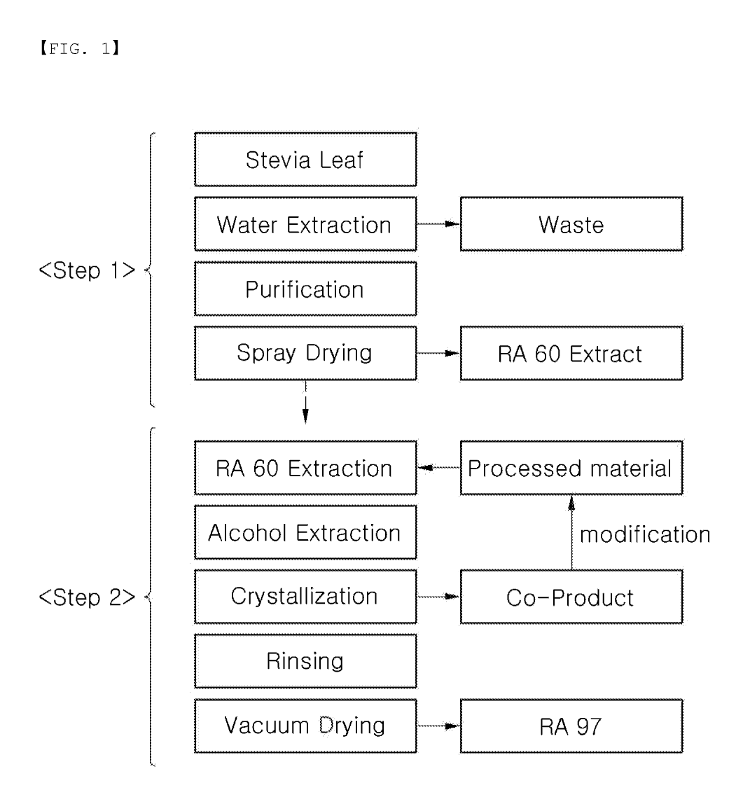

FIG. 2 shows HPLC (Agilent 1200 Series) component analysis data of a conventional RA 60 product.

FIG. 3 shows HPLC (Agilent 1200 Series) component analysis data of a sample of by-products used as a raw material in a method of producing Rebaudioside A according to an embodiment of the present invention.

### DETAILED DESCRIPTION OF THE ILLUSTRATED EMBODIMENTS

The invention is described more fully hereinafter with reference to the accompanying drawings, in which exemplary embodiments of the invention are shown. This invention may, however, be embodied in many different forms and should not be construed as limited to the exemplary embodiments set forth herein. Rather, these exemplary embodiments are provided so that this disclosure is thorough, and will fully convey the scope of the invention to those skilled in the art. In the drawings, the size and relative sizes of layers and regions may be exaggerated for clarity. Like reference numerals in the drawings denote like elements.

A method of producing Rebaudioside A in a high yield and high purity includes:

i) purifying by-products produced when a high purity steviol glycoside product is purified by fractional crystallization;

ii) performing an enzyme transferring reaction on the purified by-products to adjust a Rebaudioside A content in the by-products to be in a range of 50 weight (wt) % to 60 wt %; and

iii) fractional-crystallizing either the product produced in step ii) alone or a mixture of the product produced in step ii) and a high purity steviol glycoside product to produce Rebaudioside A.

The by-products in step i) may include 80 wt % or higher of steviol glycoside, preferably 90 wt % or higher of steviol glycoside. The by-products in step i) may include 40 to 50 wt % of stevioside.

According to the present invention, the enzyme transferring reaction is performed on the purified by-products at a temperature of 50° C. for about 5 hours in the presence of a β-1,3-glucosyl oligosaccharide compound, a β-1,3-glucanase capable of breaking down β-1,3 glucose in the compound, and a β-1,3-glucosyl transferase.

According to the present invention, the β-1,3-glucosyl oligosaccharide compound is a compound that is derived from a microorganism known in the art and examples thereof are curdlan and laminarin.

According to the method of the present invention, the purification of the by-products may be performed by, desalinization, microfiltration, or purification using an adsorption resin. However, the purification method is not limited thereto.

According to the method of the present invention, the β-1,3-glucosyl transferase may employ a microorganism or a corresponding enzyme itself having a β-1,3-glucosyl transferring activity, and examples of the microorganism or the enzyme having β-1,3-glucosyl transferring activity are those disclosed in U.S. Pat. No. 4,590,160.

The present invention also relates to a method of using by-products produced when a high purity steviol glycoside product is purified by fractional crystallization, in which the method includes, as a characteristic, re-circulating the by-products in a manufacturing process for Rebaudioside A after an enzyme transferring reaction is performed on the by-products.

The term “steviol glycoside product in a high purity” used herein refers to a product that is produced by extracting a solution containing steviol glycoside from stevia dry leaves by using a hydrothermal fluid, ethanol, methanol, or polyalcohols, and then purifying the solution to give a product containing at least 70 wt. % of steviol glycoside.

The term “RA 60” or “RA 97” used herein refers to a product containing 60 wt % or 97 wt % of Rebaudioside A based on the total weight of the product.

The unit “%” used herein refers to a weight percentage, unless otherwise defined.

The present invention will now be described in further detail with reference to the following examples. These examples are for illustrative purpose only and are not intended to limit the scope of the invention.

### EXAMPLES

### Example 1

Content and ash content analysis of steviol glycoside in conventional RA 60 product, and in residual by-products produced from a conventional Rebaudioside A, in particular, residual by-products as a mother liquor produced from a fractional crystallization process

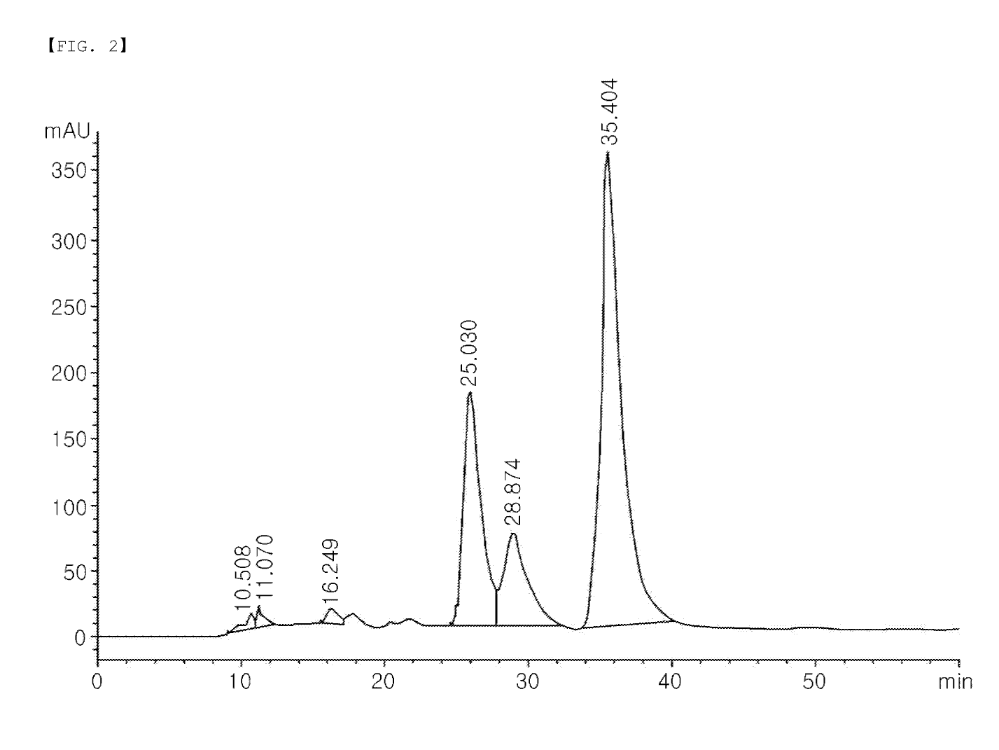

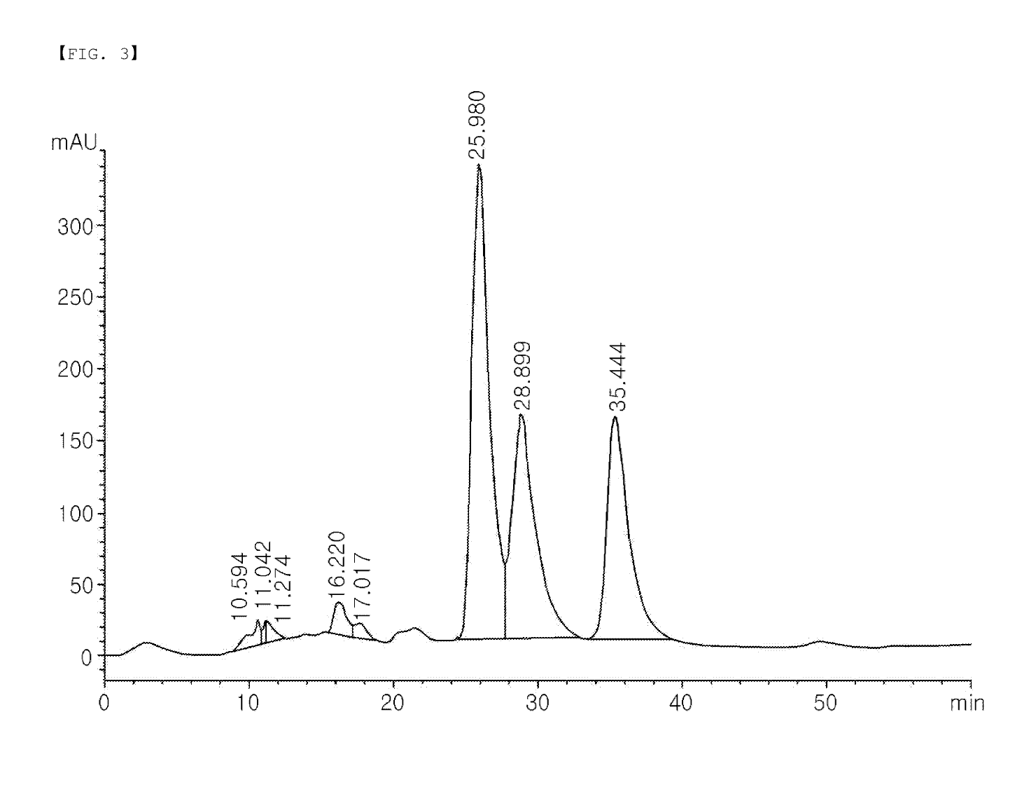

HPLC analysis was performed to identify a content of steviol glycoside. To do this, 1 g of each of the samples and 1000 ml of distilled water were loaded into a 1000 ml mass cylinder and uniformly mixed, and then filtered through a 0.45 μm aqueous filter. HPLC (Agilent 1200 Series) device was used and 20 μl of each of the samples was loaded thereto. Analysis conditions were set to be a flow rate of 0.5 ml/min and a wavelength of 210 nm. As shown in FIGS. 2 and 3, contents of stevioside, Rebaudioside A, and Rebaudioside C were measured.

As analysis results, the conventional RA60 product included 23.3% of stevioside, 61.4% of Rebaudioside A, and 12.7% of Rebaudioside C, and the by-products included 42.6% of stevioside, 25% of Rebaudioside A, and 26.9% of Rebaudioside C.

TABLE 1
Content Difference and Major Content Factor Comparison

| | | Stevioside | Rebaudioside C | Rebaudioside A |
|---|---|---|---|---|
| RA 60 | % | 23.3 | 12.7 | 61.4 |
| C0-product | % | 42.6 | 26.9 | 25.0 |

An ash content of each of the conventional RA 60 product and the by-products was analyzed. As a result, it was confirmed that the conventional RA 60 included 7% of ash and the by-products included 15% of ash.

### Example 2

### Purification of By-Products

In order to process residual by-products produced following the conventional RA60 product being manufactured, that is, by-products as a mother liquor produced from crystallization to have a reusable level, a content pattern of steviol glycoside in the by-products that had been decolorized and purified was analyzed in the same manner as in example 1 to identify contents of stevioside, Rebaudioside A, and Rebaudioside C.

After the by-products were purified, ash contents of stevioside, Rebaudioside A, and Rebaudioside C from each of processes were analyzed.

### Example 3

### Enzyme Transferring Process

By-products as a mother liquor produced from a fractional crystallization process were processed to have a reusable level as in example 2, and stevioside in the by-products was used as an acceptor and reacted in the presence of a β-1,3-glucosyl transferase, β-1,3-glucosyl oligosaccharide compound and a β-1,3-glucanase for breaking down β-1,3 glucose contained in the β-1,3-glucosyl oligosaccharide compound at a temperature of 50° C. for 5 hours, thereby producing Rebaudioside A.

A content pattern of the newly formed steviol glycoside was analyzed in the same manner as in example 1 to identify contents of stevioside, Rebaudioside A, and Rebaudioside C.

As apparent from the above description, according to the method according to the exemplary embodiments, by-products that are produced when extracted Rebaudioside A (RA) is purified and crystallized, that is, residual by-products are recycled as a raw material to increase a production yield, thereby enabling production of a product having price competitiveness. In addition, residual by-products as a mother liquor produced from a fractional crystallization process is used as a starting material and subjected to a series of processes to have a reusable level suitable for the second step process (high purity Rebaudioside A production process), thereby reforming the by-products into a high value-added product.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

What is claimed is:

1. A method of manufacturing Rebaudioside A in a high yield, the method comprising:
i) a step for purifying by-products produced when a high purity steviol glycoside product is purified by fractional crystallization;
ii) a step for performing an enzyme transferring reaction on the purified by-products to adjust a Rebaudioside A content in the by-products to be in a range of 50 weight (wt) % to 60 wt %; and
iii) fractional-crystallizing either the product produced in step ii) alone or a mixture comprising the product produced in step ii) and a high purity steviol glycoside product.

2. The method of claim 1, wherein the by-products from step i) comprise 80 wt. % or higher of stevioside glycoside.

3. The method of claim 1, wherein the enzyme transferring reaction is performed on the purified by-products in the presence of a β-1,3-glucosyl oligosaccharide compound, a β-1,3-glucanase capable of breaking down β-1,3 glucose in the compound, and a β-1,3-glucosyl transferase.

4. The method of claim 3, wherein the enzyme transferring reaction is performed at a temperature of 50° C. for 5 hours.

5. A method of using by-products produced when a high purity steviol glycoside product is purified by fractional crystallization, wherein the method comprising steps of performing an enzyme transferring reaction on the by-products; and re-circulating the by-products in a manufacturing process for Rebaudioside A.

6. The method of claim 5, wherein the enzyme transferring reaction is performed on the purified by-products in the presence of a β-1,3-glucosyl oligosaccharide compound, a β-1,3-glucanase capable of breaking down β-1,3 glucose in the compound, and a β-1,3-glucosyl transferase.
